(19) United States
(12) Patent Application Publication
(10) Pub. No.: US 2025/0067733 A1
(43) Pub. Date: Feb. 27, 2025

(54) LOW VOLUME LOGIC FOR A TEST STRIP HAVING MULTIPLE WORKING ELECTRODES

(71) Applicant: Lifescan IP Holdings, LLC, Malvern, PA (US)

(72) Inventors: Martin Lamacka, Inverness (GB); David McColl, Inverness (GB); Antony Smith, Dingwall (GB); Alexander Strachan, Murray (GB); Steven Setford, Fortrose (GB)

(73) Assignee: Lifescan IP Holdings, LLC, Malvern, PA (US)

(21) Appl. No.: 18/237,004

(22) Filed: Aug. 23, 2023

(51) Int. Cl. G01N 33/543

(52) U.S. Cl. CPC G01N 33/5438; G01N 33/54386

(57) ABSTRACT

A method for compensating an analyte measurement in the event of a low sample volume of a deposited fluid sample onto a test strip having first and second working electrodes and a reference electrode. At least one test voltage is applied between the first and second working electrodes and the reference electrode and current is measured at each of the working electrodes at a predetermined time point. The difference in measured current is compared to a stored threshold. If the difference in measured currents is within the stored threshold, the current measurements are used in determining an analyte of interest at a final test time. If the difference in measured currents is not within the stored threshold, then only the measured current of the first working electrode with a suitable correction applied is used in determining the analyte of interest at the final test time.

### TECHNICAL FIELD

This application is generally directed to the field of analyte measurement and more specifically to a glucose measurement system including a test meter and a test strip having two or more working electrodes in which the test meter is programmed in order to compensate/adjust an analyte measurement based on the determination of a lower than typical volume of a fluid sample being deposited on the test strip.

### BACKGROUND

Biosensors, such as electrochemically based test strips, are designed to measure the concentration of glucose in a physiological fluid sample from diabetic patients. The measurement of glucose can be based on the selective oxidation of glucose by the enzyme glucose oxidase (GO) on selective electrodes based on the passage of a blood sample deposited on the test strip under the application of at least one test voltage.

There are a number of known glucose measurement systems having electrochemically-based test strips that are configured for use with a test meter. In these measurement systems, glucose concentration can be determined for a subject over the course of time.

Electrochemically-based test strips may be adversely affected by the presence of certain blood components that may undesirably affect the measurement and lead to inaccuracies in the detected signal. For example, the blood hematocrit level (i.e., the percentage of the amount of blood that is occupied by red blood cells) can erroneously affect a resulting analyte concentration measurement.

Accordingly, there are test strips, for example, those manufactured by the Applicant made up of a planar substrate having a pair of working electrodes, namely a first working electrode and a second working electrode, which are disposed in coplanar and spaced relation along a fluid flow path on the test strip and in which the first and second working electrodes are of substantially the same planar size relative to one another. The test strip further includes a reference electrode, as well as a pair of physical characteristic (hematocrit) sensing electrodes, the latter also disposed in spaced relation on the substrate. Predetermined voltages applied to the electrodes produce a redox-type reaction in the presence of a deposited fluid sample to the test strip. The presence of hematocrit (a physical characteristic) can also be determined by measuring the impedance at the physical characteristic sensing electrodes in order, to enable a measured current level indicative of glucose concentration to be corrected.

For literally any test strip and in order to provide an effective result (glucose concentration), a sufficient volume of fluid (i.e., blood) must be deposited and delivered to the spaced working electrodes of the test strip. A less than adequate fill results with less surface area of an electrode being covered with sample fluid, can also lead to an inaccurate glucose determination. While there are known glucose measurement systems having test meters that are programmed to compensate a glucose measurement based on determinations of a low deposited fluid volume, there is a general and prevailing need in the field to improve the efficacy of these measurement systems.

### BRIEF DESCRIPTION

Therefore and according to a first aspect, there is provided a method for compensating an analyte measurement in the event of a low sample volume of a fluid sample being deposited onto a test strip, the test strip having at least first and second working electrodes and a reference electrode disposed in spaced relation, the method comprising:
depositing the fluid sample onto the test strip and inserting the test strip into a test meter having a processor;
using the test meter, applying at least one test voltage between the first and second working electrodes and the reference electrode;
using the processor, measuring current at each of the first and second working electrodes at a predetermined time point following a start to a test sequence;
using the processor, comparing the difference in current measured at the first working electrode and the current measured at the second working electrode at the predetermined time point relative to a stored threshold; wherein if the difference in currents measured between the first working electrode and the second working electrode at the predetermined time point is within the stored threshold, then using the current measurements of the first and second working electrodes in determining an analyte of interest at a final test time; and if the difference in measured currents between the first working electrode and the second working electrode is not within the stored threshold at the predetermined time point, then using only the measured current of the first working electrode, wherein the measured current of the first working electrode with a correction applied for purposes of determining the analyte of interest at the final test time.

According to at least one version and if the difference in measured currents between the first working electrode and the second working electrode relative to the stored threshold is not met, the processor is configured to determine two (2) final test times, namely a first final test time based on the measured currents of the first and second electrodes and a second final test time based on the corrected measured current of the first working electrode. The method further includes measuring current transients of either the first working electrode and/or the first and second working electrodes taken between the predetermined time point and a later predetermined time. The processor is further programmed to measure and store current transients until the longer of the first or second final test times. In at least one embodiment and if the difference between the currents of the first and second working electrodes exceed a predetermined ratio or if the moving average of the current differences are greater than a predetermined level, then an error is annunciated and the test sequence is terminated.

On the other hand and if the difference of measured currents between the first and second working electrode at the predetermined test time is within the predetermined threshold (which can be a range of values), then the processor of the test meter is programmed to use the currents of the first and second working electrodes and a single final test time based on the currents of both working the first and second working electrodes. According to one version, the final test time is determined based on a physical characteristic (e.g., hematocrit) of the fluid sample and two times the measured current of the first working electrode measured at a first test time. In at least one version, a look up table is stored by the test meter that can be used to determine any of the noted final test times based at least on measured currents.

A current transient (current versus time) will be measured for each of the first and second working electrodes until the determined final test time(s). At the final test time, a low fill volume logic determination will be made. This determination will examine the difference between measured currents at the first and second working electrodes at the second test time and compare the measured difference to a bias threshold. If the bias threshold is not exceeded, then the measured currents of both the first and second working electrodes will be used for the analyte concentration calculation. If the threshold is exceeded, then additional determinations are made based on the current transients of the first and second working electrodes. According to at least one embodiment, the ratio of the difference in currents of the first and second working electrodes are determined at the first and final test time. This ratio is compared to a threshold level as part of the low volume logic. According to another embodiment, the shape of the current transients are compared, for example, to a typical Cottrell current distribution. According to yet another embodiment, a moving average of the current values of the first and second working electrodes can be determined and compared to a stored threshold or range of threshold values.

According to another aspect of the present invention, there is provided an analyte measurement system comprising a test strip and a test meter, the test strip comprising a first working electrode and a second working electrode in spaced relation, the test meter including a processor configured to apply one or more test voltages to the test strip after a fluid sample has been deposited. The processor is programmed with logic to measure current levels at a predetermined time point following the initiation of a test sequence at each of the first and second working electrodes and compare a difference in the measured current values to a predetermined threshold. If the measured current difference is within the predetermined threshold at the predetermined time point, the processor is further programmed to determine a single final test time based on the measured current of the first and second working electrodes and if the threshold is not met then the processor is configured to determine two (i.e., second) test times, one of the final test times being based on both the first and second working electrode currents at the predetermined time point and the other final test time being based on a suitably corrected measured current of the first working electrode at the predetermined time point.

According to at least one embodiment, the final test time(s) are determined based on the measured currents of the first and second working electrodes at the first test time and a physical characteristic of the fluid sample deposited on the test strip. According to at least one version, the physical characteristic is hematocrit of a deposited blood sample. In at least one version, the test strip further comprises physical characteristic sensing electrodes. In at least one embodiment, the second test times can be determined using both the measured current of the first and second working electrodes and a measured impedance of the physical characteristic sensing electrodes using an algorithm or alternatively, a look-up table stored by the processor.

Various shape and bias checks are made in regard to the measured current transients of the first and second working electrodes. For example, a ratio can be determined by measuring the difference in measured currents between the first and second working electrodes at a predetermined time point during the testing sequence and measured currents between the first and second working electrodes at a final test time. This ratio can be compared to a predetermined threshold that is stored by the test meter. According to another version, a moving average of the measured current values along the transient can be determined and compared to a stored threshold or range of threshold values. According to yet another exemplary version, a shape check can be made between a measured current transient and a typical Cottrell current distribution.

One advantage that is realized by the present invention is that negative test bias caused by lower volumes of an applied fluid sample can be accounted for and significantly reduced by the herein described analyte measurement system and related method.

Another advantage is that glucose measurements can still be successfully obtained using lower volumes of applied fluid sample than in prior art analyte measurement systems, which would ordinarily be insufficient for testing purposes.

These and other features and advantages will be readily apparent from the following Detailed Description, which should be read in conjunction with the accompanying drawings.

### BRIEF DESCRIPTION OF THE DRAWINGS

The accompanying drawings, which are incorporated herein and constitute part of this specification, illustrate presently preferred embodiments of the invention, and together with the general description given above and the detailed description given below, serve to explain features of the invention (in which like numerals represent like elements), of which:

FIG. 1A illustrates an exemplary analyte measurement system that includes a biosensor (test strip) and a test meter;

FIG. 1B depicts yet another exemplary analyte measurement system that includes a biosensor (test strip) and a test meter;

FIG. 2A illustrates in simplified schematic form, the components of a test meter used in the analyte measurement system;

FIG. 2B depicts a simplified schematic diagram of an implementation of the test meter of FIG. 2A;

FIG. 2C depicts a simplified block diagram of various blocks of the test meter of FIGS. 1A and 1B;

FIG. 2D depicts a simplified block diagram of a physical characteristic measurement block;

FIG. 3A illustrates a test strip for the analyte measurement system of FIGS. 1A and 1B;

FIG. 3B is a perspective view of an alternate test strip (biosensor) for the analyte measurement system of FIGS. 1A and 1B;

FIG. 3C is a perspective view of variations of the test strip (biosensor) of FIGS. 3A and 3B;

FIG. 4A is a graph of time over an applied potential for the biosensor (test strip) of FIG. 3A, 3B, or 3C in which and for illustrative purposes, a five (5) second test time is shown;

FIG. 4B is a graph of time over current output for the biosensor (test strip) of FIG. 3A, 3B or 3C in which and for illustrative purposes, a five (5) second test time is shown;

FIGS. 5A-5C diagrammatically presents various fluid fill conditions of a biosensor (test strip) having at least two working electrodes;

FIGS. 6(a) and 6(b) depict differences in measured current transients of a biosensor (test strip) having two working electrodes based on a complete fill and an incomplete fill, respectively, per the fill conditions shown in FIGS. 5A and 5B;

FIG. 7 is a flow chart of an exemplary method for determining and compensating for incomplete fill volumes of a test strip in accordance with various aspects of the present invention;

FIGS. 8(a) and 8(b) depict one exemplary technique for verifying the integrity of the current transients of the first and second working electrodes of a test strip as taken between first and second times of a test sequence, and more specifically determining a ratio bias;

FIGS. 9(a) and 9(b) depicts another exemplary technique for verifying the integrity of current transients of the first and second working electrodes of a test strip based on shape biasing as compared to predetermined (Cottrell) transient;

FIG. 10 depicts an exemplary logic matrix based on the determinations of measured currents between the first and second working electrodes of a test strip as well as determinations of shape and ratio biasing; and

FIGS. 11(a) and 11(b) depict another alternative and exemplary technique for determining the integrity of current transients of the first and second working electrodes of the test strip using a moving average.

### DETAILED DESCRIPTION OF PREFERRED EMBODIMENTS

The herein described invention relates to exemplary embodiments of an analyte (glucose) measurement system minimally including a biosensor (test strip) having two or more spaced working electrodes and a test meter that is configured to receive the test strip. The herein described invention further describes a related method in which an analyte measurement can be compensated or adjusted based on a determination of low fluid volume to at least one of the working electrodes. The following detailed description should be read in reference to the accompanying drawings, in which like elements in different drawings are identically numbered. The drawings, which are not necessarily to scale, depict selected embodiments and are not limited to the scope of the invention. The detailed description illustrates, by way of example, not by way of limitation, the principles of the invention. This description will clearly enable one skilled in the art to make and use the invention, and describes several embodiments, adaptations, variations, alternatives and uses of the invention, including what is believed to be the best mode of carrying out the invention.

As used herein, the terms “about” or “approximately” for any numerical values or ranges indicate a suitable dimensional tolerance that allows the part or collection of components to function for the intended purpose as described herein. More specifically, “about” or “approximately” may refer to the range of values±10 percent of the recited value, e.g., “about 90%” may refer to the range of values from 810% to 99%. In addition and as used herein, the terms “patient”, “host”, “user”, “person” and “subject” refer to any human or animal subject and are not intended to limit the systems or methods to human use, although use of the subject invention in a human patient represents a preferred embodiment. As used herein, “oscillating signal” includes voltage signal(s) or current signal(s) that, respectively, change polarity or alternate direction of current or are multi-directional. As used here, the term “electrical signal” or “signal” is intended to include direct current signal, alternating signal or any signal within the electromagnetic spectrum. The terms “processor”, “microprocessor” or “microcontroller” are intended to have the same meaning and are intended to be used interchangeably, as are the terms “biosensor” and “test strip” for purposes of this discussion.

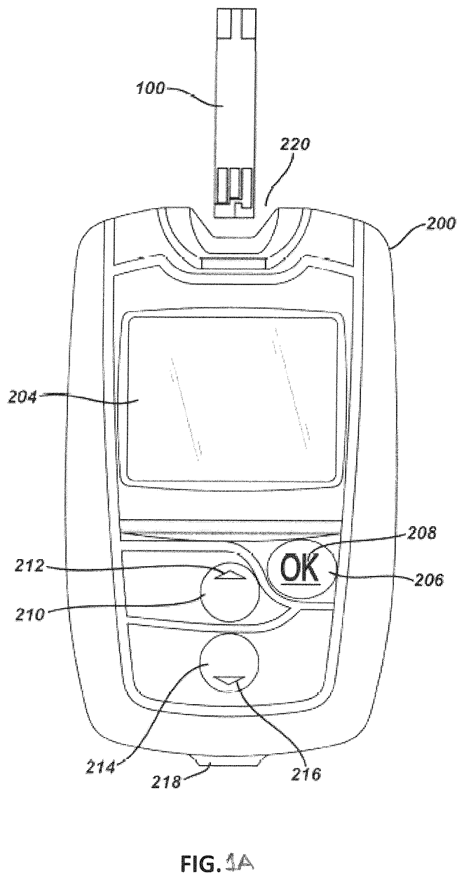

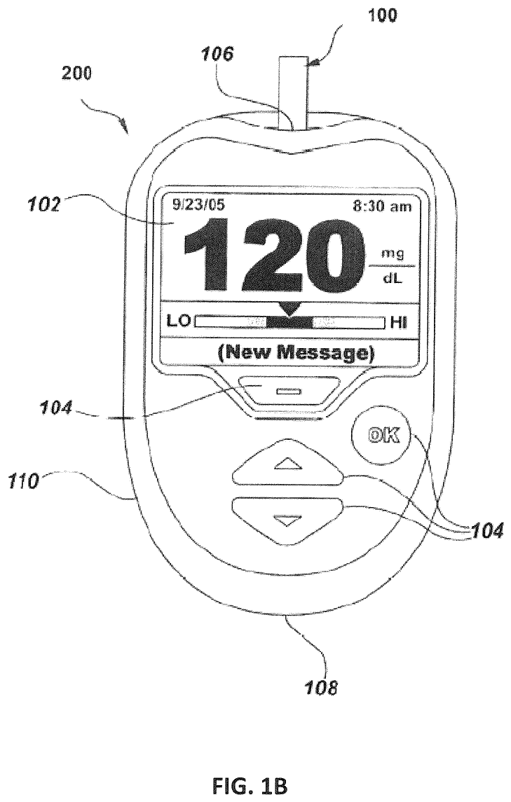

FIG. 1A illustrates a glucose measurement system having one or more biosensors (e.g., test strips) 100 (only a single test strip being depicted in this view) and a test meter 200 for testing analyte (e.g., glucose) levels in the blood of an individual using a biosensor 100 produced by the methods and techniques illustrated and described herein. The test meter 200 may include user interface inputs, namely 206, 210, 214, which can be in the form of buttons, for entry of data, navigation of menus, and execution of commands. Data can include values representative of analyte (glucose) concentration, and/or information that are related to the everyday lifestyle of an individual. Information, which is related to the everyday lifestyle, can include food intake, medication use, the occurrence of health check-ups, general health condition and exercise levels of an individual. The test meter 200 can also include a display 204 that can be used to report measured glucose levels, and to facilitate entry of lifestyle related information.

The test meter 200 may include a first user interface input 206, a second user interface input 210, and a third user interface input 214, though it will be understood that the specific number and form of interface inputs can be suitably varied. The user interface inputs 206, 210 and 214 facilitate entry of data stored in the test meter, enabling a user to navigate through the user interface displayed on the display 214. The user interface inputs 206, 210 and 214 include a first marking 208, a second marking 212, and a third marking 216, which help in correlating user interface inputs to characters on the display 204.

The test meter 200 can be activated by inserting a biosensor 100 (or its variants) into a strip port connector 220, by pressing and briefly holding the first user interface input 206, or by the detection of data traffic across a data port 218. The test meter 200 can be switched off by removing the biosensor 100, pressing and briefly holding the first user interface input 206, navigating to and selecting a meter off option from a main menu screen, or by not pressing any buttons for a predetermined time. The display 204 can optionally include a backlight (not shown).

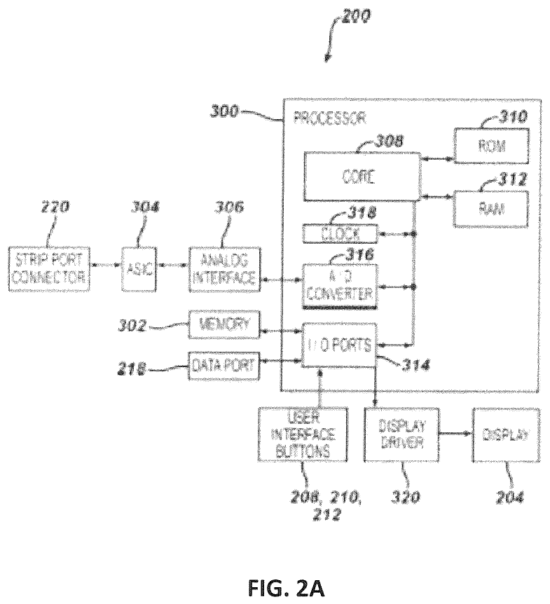

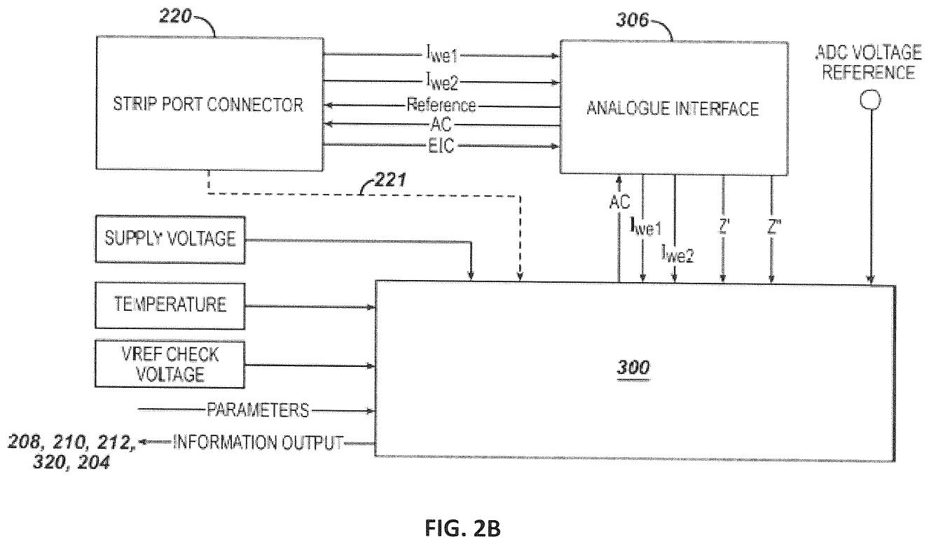

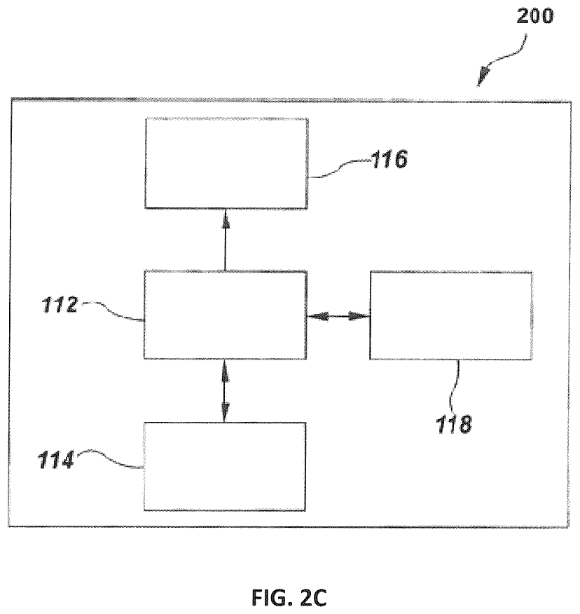

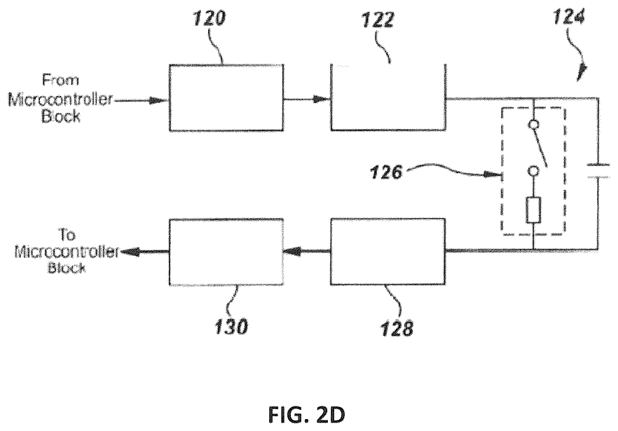

Referring to FIG. 2A, an internal layout of an exemplary test meter 200 is shown. The test meter 200 may include a resident processor 300 The processor 300 can be bi-directionally connected via I/O ports 314 to a memory 302, which in some embodiments can be an EEPROM. Also connected to the processor 300 via the I/O ports are the data port 218, the user interface inputs 206, 210, 214 and a display driver 320. The data port 218 can be connected to the processor 300, thereby enabling transfer of data between the memory 302 and an external device, such as a personal computer. The user interface inputs 206, 210 and 214 are directly connected to the processor 300. The processor 300 controls the display 204 via the display driver 320. The memory 302 can be pre-loaded with calibration information, such as batch slope and batch intercept values, during the production of the test meter 200. This pre-loaded calibration information can be accessed and user by the processor 300 upon receiving a suitable signal (such as current) from the biosensor via the strip port connector 220 so as to calculate a corresponding analyte level (such as blood glucose concentration) using the signal and the calibration information without receiving calibration input from any external source.

In embodiments described and illustrated herein, the test meter 200 may include an Application Specific Integrated Circuit (ASIC) 304, so as to provide electronic circuitry used in measurements of glucose level in blood that has been applied to a test strip 100 (or its variants) inserted into the strip port connector 220. Analog voltages may pass to and from the ASIC 304 by way of an analog interface 306. Analog voltages from analog interface 306 can be converted into digital signals by an A/D converter 316. The processor 300 further includes a core 308, a ROM 310 (containing computer code), a RAM 312, and a clock 318. In at least one embodiment, the processor 300 is configured (or programmed) to disable all of the user interface inputs except for a single input upon a display of an analyte value by the display unit such as, for example, during a time period after an analyte measurement. In an alternative embodiment, the processor 300 is configured (or programmed) to ignore any input from all of the user interface inputs except for a single input upon a display of an analyte value by the display unit. Detailed descriptions and illustrations of the test meter 200 are shown and described in International Patent Application Publication No. WO 2006/6070200, which is incorporated by reference into this application as if fully set forth herein.

Referring to FIG. 1B, there is shown another embodiment of a hand-held test meter 200. This version of the test meter 200 includes a display 102, a plurality of user interface buttons 104, a strip port connector 106, a USB interface 108, and a housing. Referring to FIGS. 2A-2D, the hand-held test meter 200 of FIGS. 1A and 1B also commonly includes a microcontroller block 112, a physical characteristic measurement block 114, a display control block 116, a memory block 118 and other electronic components (not shown) for applying a test voltage to a biosensor, such as test strip 100, and also for measuring an electrochemical response (e.g., a plurality of test current values) and determining an analyte (e.g., glucose) based on the electrochemical response. To simplify the present descriptions, the FIGURES do not depict all such electronic circuitry.

The display 102 can be, for example, a liquid crystal display or a bi-stable display configured to show a screen image. An example of a screen image may include a glucose concentration, a date and time, an error message, and a user interface for instructing an end user how to perform a test sequence.

The strip port connector 106 is configured to operatively interface with a biosensor 100, such as an electrochemical-based test strip (or its variants) configured for the determination of glucose in a whole blood sample. Therefore, the biosensor is configured for operative insertion into the strip port connector 106 and in at least some embodiments to operatively interface with phase-shift-based hematocrit measurement block 114 via, for example, suitable electrical contacts (not shown).

The USB interface 108 can be any suitable interface known to those skilled in the art. The USB interface 108 is essentially a passive component that is configured to power and provide a data line to the hand-held test meter 200.

Once a biosensor 100 is interfaced with the test meter 200 or prior thereto, a bodily fluid sample (e.g., a whole blood sample) is introduced into a sample chamber of the biosensor. The biosensor can include enzymatic reagents that selectively and quantitatively transform an analyte into another predetermined chemical form. For example, the biosensor can include an enzymatic reagent with ferricyanide and glucose oxidase so that glucose can be physically transformed into an oxidized form.

The memory block 118 of the hand-held test meter 200 includes a suitable algorithm and can be configured, along with the microcontroller block 112 to determine an analyte based on the electrochemical response of the biosensor and according to at least some embodiment, the hematocrit level of the introduced fluid sample. For example, in the determination of the analyte blood glucose, the hematocrit can be determined to compensate for the effect of hematocrit on electrochemically determined blood glucose concentrations.

The microcontroller block 112 is disposed within the housing of the test meter 200 and can include any suitable microcontroller and/or micro-processor known to those of skill in the art. According to at least one embodiment, the microcontroller can generate a square wave of 25 to 250 kHz and a 90 degree phase-shifted wave of the same frequency, and thereby function as a signal generation s-block. The microcontroller may also have Analog to Digital (A/D) processing capabilities suitable for measuring voltages generated by phase shift based hematocrit measurement blocks which may be employed in some embodiments.

Referring in particular to FIG. 2D, a phase-shift-based hematocrit measurement block 114 includes a signal generation sub-block 120, a low pass filter sub-block 122, a biosensor sample cell interface sub-block 126 (within the dashed lines of FIG. 2D), a transimpedance amplifier sub-block 128, and a phase detector sub-block 130.

The phase-shift-based hematocrit measurement block 114 and microcontroller block 112 are configured to measure the phase shift of a bodily fluid sample in a sample cell of a biosensor inserted in the hand-held test meter by, for example, measuring the phase shift of one or more high frequency electrical signals driven through the bodily fluid sample. In addition, the microcontroller block 112 is configured to compute the hematocrit of the bodily fluid based on the measured phase shift. The microcontroller block 112 can compute the level of hematocrit by, for example, employing an A/D converter to measure voltages received from a phase-detector sub block, convert the measured voltages into a phase-shift and then employing a suitable algorithm or look-up table to convert the phase-shift into a hematocrit value. Such an algorithm and/or look-up table will be configured suitably to take into account various factors such as strip geometry (including the area of the electrode(s) and sample chamber volume) and signal frequency.

It has been determined that a relationship exists between the reactance of a whole blood sample and the hematocrit of the sample. Electrical modeling of a bodily fluid sample (i.e., a whole blood sample) as parallel capacitive and resistive components indicates that when an alternating current (AC) signal is forced through the bodily fluid sample, the phase shift of the AC signal will be dependent on both the frequency of the AC voltage and the hematocrit level of the sample. Moreover, modeling indicates that hematocrit has a relatively minor effect on the phase shift when the frequency of the signal is in the range of approximately 10 kHz to 25 kHz and a maximum effect on the phase shift when the frequency of the signal is in the range of approximately 250 kHz to 500 kHz. Therefore, the hematocrit of a bodily fluid sample can be measured by, for example, driving AC signals of known frequency through the bodily fluid sample and detecting their phase shift. For example, the phase shift of a signal with a frequency in the range of 10 kHz to 25 kHz can be used as a reference reading in such a hematocrit measurement while the phase shift of a signal with a frequency in the range of 250 kHz to 500 kHz can be used as the primary measurement.

Additional details relating to features pertaining to the physical characteristic measurement blocks and use of the physical characteristic (hematocrit) measuring electrodes of the biosensor are provided in U.S. Pat. No. 9,423,374 B2, which is incorporated in its entirety herein.

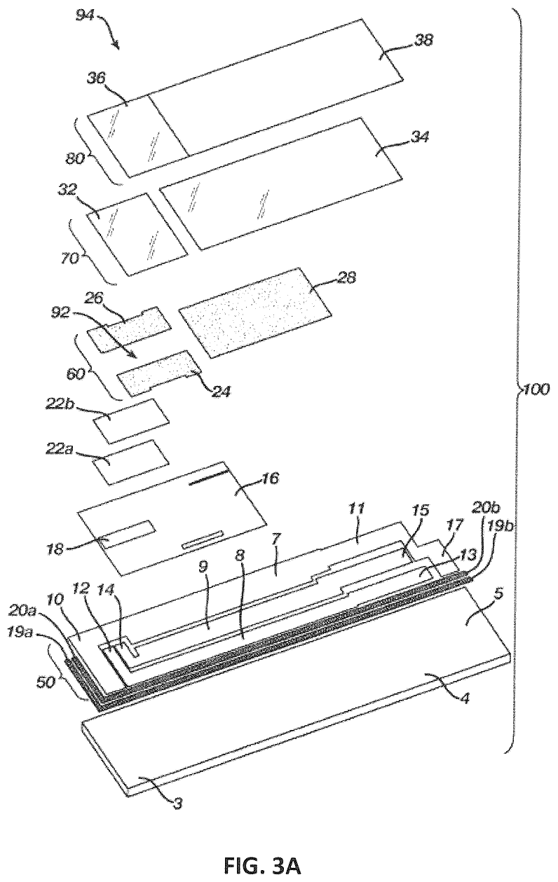

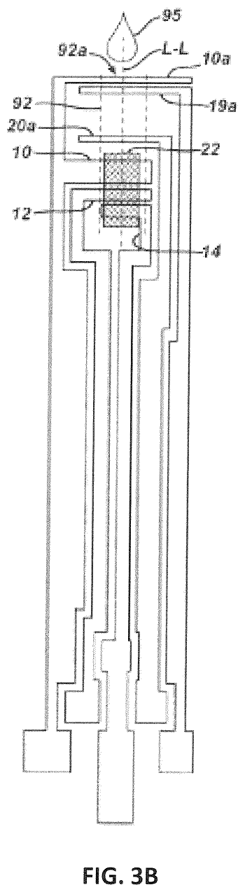

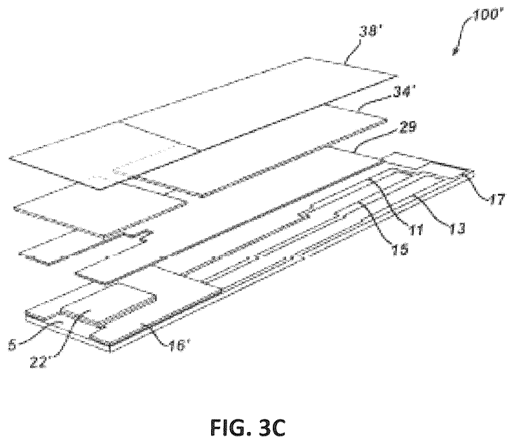

FIG. 3A is an exploded perspective view of an exemplary test strip 100, which may include seven layers disposed on a substrate 5. The seven layers disposed on the substrate 5 may be a conductive layer 50 (which can also be referred to as an electrode layer 50), an insulation layer 16, two overlapping reagent layers 22a and 22b, an adhesive layer 60 which includes adhesive portions 24, 26 and 28, a hydrophilic layer 70 which may include portions 32, 34, and a top layer 80 having portions 36, 38 which forms a cover for the test strip 100. The test strip 100 may be manufactured in a series of steps in which the conductive layer 50, insulation layer 16, reagent layers 22, and adhesive layer 60 are sequentially deposited on the substrate 5 using, for example, a screen printing process. Note that the electrodes 10, 12, and 14 are disposed for contact with the reagent layer 22a and 22b whereas the physical characteristic electrodes 19a and 20a are spaced apart according to this particular embodiment and not in contact with the reagent layer 22. The hydrophilic layer 70 and the top layer 80 may be disposed from a roll stock and laminated onto the substrate 5 as either an integrated laminate or as separate layers. It is noted that the reagent includes both the enzymes and other materials, such as binders and other materials, to allow the reagent to function for its intended purpose in a biosensor. The test strip 100 has a distal portion 3 and a proximal portion 4, as shown in FIG. 3A.

The test strip 100 may include a sample receiving chamber 92 through which a physiological fluid sample 95 may be drawn through or deposited (FIG. 3B). The physiological fluid sample discussed herein may be blood. The sample receiving chamber 92 may include an inlet at a proximal end of the test strip 100 and an outlet at the side edges of the test strip 100, as illustrated in FIG. 3A. A fluid sample 95 can be applied to the inlet along an axis L-L (FIG. 3B) to fill the sample receiving chamber 92 so that glucose can be measured. The side edges of a first adhesive pad 24 and a second adhesive pad 26 located adjacent to reagent layer 22 each define a wall of the sample receiving chamber 92, as illustrated in FIG. 3A. A bottom portion or “floor” of the sample receiving chamber 92 may include a portion of the substrate 5, conductive layer 50, and insulation layer 16, as illustrated in FIG. 3A. A top portion or “roof” of the sample receiving chamber 92 may include a distal hydrophilic portion 32 of the hydrophilic layer 70, as illustrated in FIG. 3A.

For the test strip 100, as illustrated in FIG. 3A, the substrate 5 may be used a foundation for assisting support of subsequently applied layers of the test strip 100. The substrate 5 may be in the form of a polyester sheet such as a polyethylene tetraphthalate (PET) material. The substrate 5 can be in a roll format, nominally 350 microns thick by 370 millimeters wide and approximately 600 meters in length.

A conductive layer may be required for forming electrodes that may be used for the electrochemical measurement of glucose. The first conductive layer 50 may be made from a carbon ink that is screen-printed onto the substrate 5. In a screen-printing process, carbon ink is loaded onto a screen and then transferred through the screen using a squeegee. The printed carbon ink may be dried using hot air at about 140° C. The carbon ink may include resin, carbon black and graphite and one or more solvents for the resin, carbon and graphite mixture. More particularly, the carbon ink may incorporate particular ratios of carbon black:resin and carbon black:graphite in the carbon ink.

For the exemplary test strip 100, as illustrated in FIG. 3A, the first conductive layer 50 may include a reference electrode 10, a first working electrode 12, a second working electrode 14, third and fourth physical characteristic sensing electrodes 19a and 20a, a first contact pad 13, a second contact pad 15, a reference contact pad 11, a first working electrode track 8, a second working electrode track 9, a reference electrode track 7, and a strip detection bar 17. The physical characteristic sensing electrodes 19a and 20a are provided with respective electrode tracks 19b and 20b. The conductive layer may be formed from carbon ink. The first contact pad 13, the second contact pad 15 and the reference contact pad 11 may be adapted to electrically connect to a test meter, such as meter 200, FIG. 2A. The first working electrode track 8 provides an electrically continuous pathway from the first working electrode 12 to the first contact pad 13. Similarly, the second working electrode track 9 provides an electrically continuous pathway from the second working electrode 14 to the second contact pad 15 and the reference electrode track 7 provides an electrically continuous pathway from the reference electrode 10 to the reference contact pad 11. The strip detection bar 17 is electrically connected to the reference contact pad 11. The third and fourth electrode tracks 19b and 20b connect to the respective electrodes 19a and 20a. A test meter such as meter 200, FIG. 2A, may detect that the test strip 100 has been properly inserted t by measuring a continuity between the reference contact pad 11 and the strip detection bar 17, as illustrated in FIG. 3A.

In the embodiment of FIG. 3B, which is a variation of the test strip of FIG. 3A, an additional electrode 10a is provided as an extension of any of the electrodes 19a, 20a, 14, 12, 10. It must be noted that the built-in shielding of grounding electrode 10a is used to reduce or eliminate any capacitance coupling between the finger or body of the user and the physical characteristic measurement electrodes 19a and 20a. To do this, the grounding electrode 10a can be connected to any of the other five electrodes according to this embodiment or to its own separate contact pad (and electrode track) for connection to ground on the meter instead of one or more of the contact pads 15, 17, 13 via respective tracks 7, 8 and 9. In one embodiment, the grounding electrode 10a is connected to one of the three electrodes that has the reagent 22 disposed thereon. For example, the grounding electrode 10a is connected to the electrode 10. Being the grounding electrode, it is advantageous to connect the grounding electrode 10a to the reference electrode 10 so not to contribute any additional current to the working electrode measurements which may come from background interfering compounds in the deposited fluid sample. Further to connecting the shield or grounding electrode 10a to the reference electrode 10, this is believed to effectively increase the size of the counter electrode which can become limiting especially at high signals. In the embodiment depicted in FIG. 3B, the reagent is disposed so it is not in contact with either of the physical characteristic sensing electrodes 19a, 20a. Alternatively, the reagent can be disposed so that the reagent contacts at least one of the sensing electrodes 19a and 20a.

In yet another alternate version of the test strip 100, shown in FIG. 3C, the top layer 38, hydrophilic film layer 34 and a spacer 29 have been combined together to form an integrated assembly for mounting to the substrate 5 with reagent layer 22′ disposed proximate the insulation layer 16′.

In the embodiment of FIG. 3B, the analyte measurement electrodes 10, 12 and 14 are disposed in generally the same configuration as the configuration shown in FIG. 3A. The electrodes 19a and 20a used to sense a physical characteristic (hematocrit) level of the deposited fluid (blood) sample, however, are disposed in a spaced apart configuration in which one electrode 19a is proximate an entrance 92a to the test chamber 92 and another electrode 20a is at an opposite end of the test chamber 92. Electrodes 10, 12 and 14 are disposed to be in contact with a reagent layer 22 whereas the physical characteristic sensing electrodes 19a and 20a are not in contact with the reagent.

In FIGS. 3A-3C, the physical characteristic (e.g., hematocrit) sensing electrodes 19a and 20a are disposed adjacent each other and can be placed at the opposite end 92b of the entrance 92a of the test chamber 92 (FIGS. 3C and 3D) or adjacent the entrance 92a (not shown for brevity). In all of these embodiments, the physical characteristic sensing electrodes are spaced apart from the reagent layer 22 so that these physical characteristic sensing electrodes are not impacted by the electrochemical reaction of the reagent in the presence of the fluid sample (e.g., blood, control solution or interstitial fluid) containing glucose.

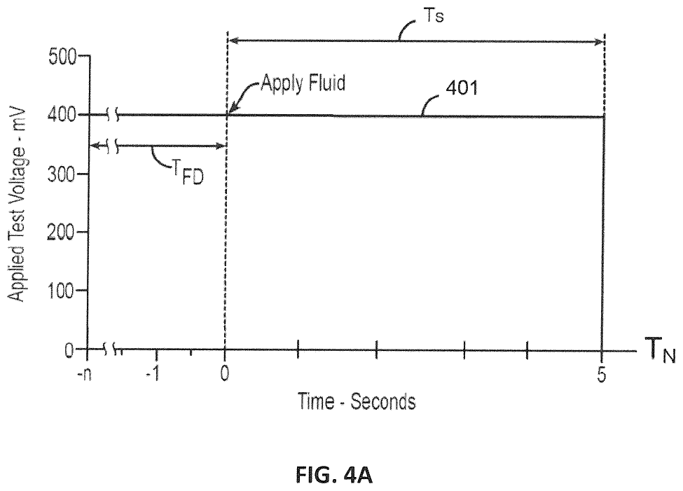

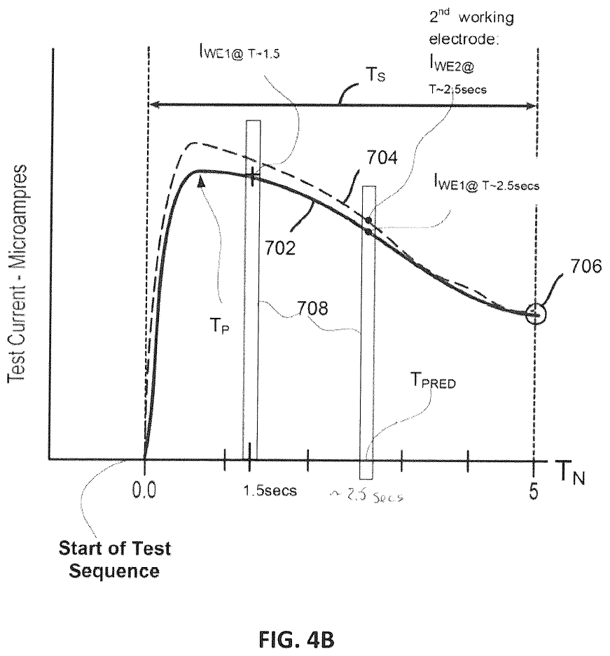

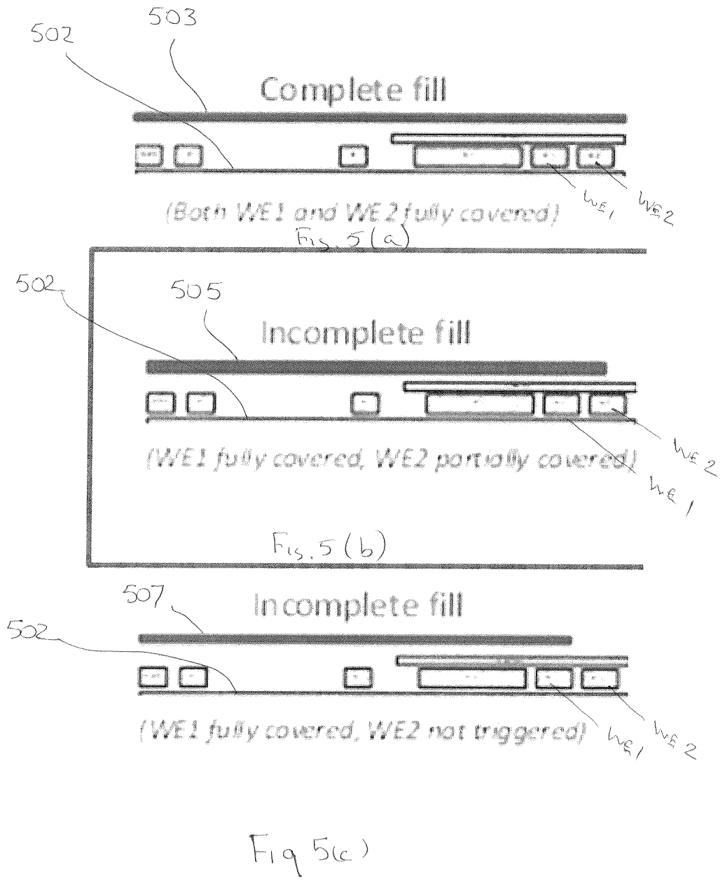

According to one or more embodiments of the biosensor (test strip or variants), there are two measurements that can be made to a fluid sample deposited on the biosensor. One of the measurements is that of the concentration of the analyte (e.g., glucose) in the fluid sample, while the other measurement is that of the physical characteristic (e.g., hematocrit) of the deposited fluid sample. The measurement of the physical characteristic (e.g., hematocrit) is used to modify or correct the glucose measurement so as to remove or reduce the effect of red blood cells on the glucose measurements. Both measurements (glucose and hematocrit) can be performed in sequence, simultaneously or overlapping in duration. For example, the glucose measurement can be performed first followed by the physical characteristic measurement, the physical characteristic (e.g., hematocrit) measurement followed by the glucose measurement, or a duration of one measurement may overlap a duration of the other measurement. Each measurement is discussed in further detail in FIGS. 4A, 4B and 5.

FIG. 4A is an exemplary chart of a test signal applied to the test strip 100 and its variations, as previously described according to FIGS. 3A-3C. Before a fluid sample is applied to the test strip 100, the test meter 200 is in a fluid detection mode in which a first test signal of about 400 millivolts applied between the second working electrode (electrode 14 of test strip 100) and the reference electrode (electrode 10) of the test strip 100. A second test signal of about 400 millivolts is preferably applied between the first working electrode (electrode 12 of test strip 100) and the reference electrode (electrode 10) of the test strip 100. Alternatively, the second test signal may also be applied contemporaneously such that a time interval of the application of the first test signal overlaps with a time interval in the application of the second test voltage. The test meter 200 may be in a fluid detection mode during a fluid detection time interval T_{FD} prior to the detection of physiological fluid at starting time at zero. In the fluid detection mode, the test meter 200 determines when a fluid sample is applied to the test strip 100 (or its variants) such that the fluid wets either the first working electrode 12 or the second working electrode 14 (or both working electrodes 12, 14) with respect to the reference electrode 10. Once the test meter 200 recognizes that the physiological fluid has been applied to the test strip 100 (or its variants) because of, for example, a sufficient increase in the measured test current at either or both of the first working electrode 12 and second working electrode 14, the test meter assigns a zero second marker at zero time “0” and starts the test time interval T_. The test meter 200 may sample the current transient signal at a suitable sampling rate, such as, for example, every 1 milliseconds to every 100 milliseconds. Upon the completion of the first test time interval T_, the test signal is removed. For simplicity FIG. 4A only shows the first test signal applied to the test strip 100 (or its variants).

Hereafter, a description of how analyte (e.g., glucose) concentration is determined from the known signal transients (e.g., the measured electrical signal response in nanoamperes as a function of time) that are measured when the test voltages of FIG. 4A are applied to the biosensor 100 (or its variants).

In FIG. 4A, the first and second test voltages applied to the biosensor (test strip 100 or its variants as discussed herein) are generally from about +100 millivolts to +600 millivolts. In one embodiment in which the electrodes include carbon ink and the mediator includes ferricyanide, the test signal is about +400 millivolts. Other mediator and electrode material combinations will require different test voltages, as is known to those skilled in the art. The duration of the test voltages is generally from about 3 seconds to about 7 seconds and is typically about 5 seconds. Typically, the test sequence Tₛ is measured relative to t₀. As the voltage 401 is maintained in FIG. 4A for the duration of Tₛ, output signals are generated, shown in FIG. 4B with the current transient 702 for the first working electrode 12 being generated starting at zero time and likewise the current transient 704 for the second working electrode 14 is also generated with respect to the zero time. It is noted that while the signal transients 702 and 704 have been placed on the same referential zero point for purposes of explaining the process, in physical terms, there is a slight time differential between the two signals due to fluid flow in the chamber towards each of the working electrodes 12 and 14 of the test strip 100 along axis L-L. However, the current transients are sampled and configured in the microcontroller to have the same start time. In FIG. 4B, the current transients build up to a peak proximate peak time T_{P} at which time, the current drops off slowly until approximately one of about 2.54 seconds and one on average about 5 seconds after zero time. At the point 706, which is on average approximately 5 seconds from the start of the test sequence, the output signal for each of the first and second working electrodes 12, 14 may be measured and added together if the sample volume is sufficient. Additional detail is discussed in a later portion concerning issues if the test fluid deposited is insufficient. For example and according to one known algorithm, the signal from only one of the working electrodes 12 and 14 may be doubled in the event of insufficient sample volume.

Referring back to FIG. 2B, the system drives a signal to measure or sample the output signal I_{E} from at least one of the working electrodes 12, 14 at any one of a plurality of time points or positions T₁, T₂, T₃ . . . T_{N}. As can be seen in FIG. 4B, the time position can be any time point or interval in the test sequence Tₛ. For example, the time position at which the output signal is measured can be a single time point T at 1.5 seconds or an interval 708 (e.g., interval—10 milliseconds or more depending on the sampling rate of the system) overlapping the time point T_{2.8} at about 2.8 seconds, according to one example.

Output transients 702 and 704 can be sampled to derive signals I_{E} (by summation of each of the current I_{WE1} and I_{WE2} or by doubling of one of I_{WE1} or I_{WE2}) at various time positions during the test sequence.

A more full description of the determination of impedance and the physical characteristic of the sample (i.e., hematocrit) is more fully described in U.S. Pat. No. 9,423,374 B2, which was previously incorporated by reference in its entirety herein.

FIG. 3A is an exemplary exploded view of yet another test strip 100″, which may include seven layers disposed on a substrate 5. The seven layers disposed on the substrate 5 can be a first conductive layer 50 (which can also be referred to as electrode layer 50), an insulation layer 16, two overlapping reagent layers 22a and 22b, an adhesive layer 60 which includes adhesive portions 24, 26 and 28, a hydrophilic layer 70, and a top layer 80 which forms a cover 94 for the test strip 100″. Test strip 100″ may be manufactured in a series of steps wherein the conductive layer 50, insulation layer 16, reagent layers 22a and 22b, and the adhesive layer 60 are sequentially deposited on the substrate 5 using, for example, a screen-printing process. Note that the electrodes 10, 12 and 14 according to this version are disposed for contact with the reagent layer 22a and 22b, whereas a pair of physical characteristic sensing electrodes 19a and 20a are spaced apart and not in contact with the reagent layer 22a and 22b. The hydrophilic layer 70 and the top layer 80 can be disposed from a roll stock and laminated onto the substrate 5 as either an integrated laminate or as separate layers. The test strip 100″ has a distal portion 3 and a proximal portion 4.

The test strip 100″ may include a sample receiving chamber 92 through which a physiological fluid sample 95 may be drawn through or deposited as shown in FIG. 3B. The physiological fluid sample discussed herein may be blood. Sample receiving chamber may include an inlet at a proximal end and an outlet at side edges of the test strip 100″, as illustrated in FIG. 3A. The physiological fluid sample 95 can be applied to the inlet along axis L-L (FIG. 3B) to fill a sample receiving chamber 92 so that glucose can be measured. The side edges of a first adhesive portion 24 and a second adhesive portion 26 located adjacent to the reagent layer 22a and 22b each define a wall of the sample receiving chamber 92, as illustrated in FIG. 3A. A bottom portion or “floor” of the sample receiving chamber 92 may include a portion of the substrate 5, conductive layer 50, and insulation layer 16, as illustrated in FIG. 3A. A top portion or “roof” of the sample receiving chamber 92 may include a distal hydrophilic portion 32, as illustrated in FIG. 3A. For the test strip 100″, as illustrated in FIG. 3A, the substrate 5 can be used as a foundation for helping support subsequently applied layers. The substrate 5 can be in the form of a polyester sheet such as a polyethylene tetraphthalate (PET) material. The substrate 5 can be in a roll format, nominally 350 microns thick by 370 millimeters wide and approximately 600 meters in length.

For the test strip 100″, as illustrated in FIG. 3A, the first conductive layer 50 may include a reference electrode 10, a first working electrode 12, a second working electrode 14, third and fourth physical characteristic sensing electrodes 19a and 20a, a first contact pad 13, a second contact pad 15, a reference contact pad 11, a first working electrode track 8, a second working electrode track 9, a reference electrode track 7, and a strip detection bar 17. The physical characteristic sensing electrodes 19a and 20a are provided with respective electrode tracks 19b and 20b. The conductive layer 50 may be formed from carbon ink. The first contact pad 13, second contact pad 15 and the reference contact pad 11 may be adapted to electrically connect to a test meter. The first working electrode track 8 provides an electrically continuous pathway from the first working electrode 12 to the first contact pad 13. Similarly, the second working electrode track 9 provides an electrically continuous pathway from the second working electrode 14 to the second contact pad 15 and the reference electrode track 7 provides an electrically continuous pathway from the reference electrode 10 to the reference contact pad 11. The strip detection bar 17 is electrically connected to the reference contact pad 11. The third and fourth electrode tracks 19b and 20b connect to the respective physical characteristic sensing electrodes 19a and 20a. A test meter may detect that the test strip 100″ has been properly inserted t by measuring a continuity between the reference contact pad 11 and the strip detection bar 17, as illustrated in FIG. 3A.

With reference to FIGS. 5(a)-5(b), the determination of a low volume condition in accordance with the prior procedure is described for the foregoing analyte measurement system. More specifically, a “low volume event” is depicted for a test strip 502, having features as previously described, including a pair of spaced working electrodes WE₁ and WE₂. As shown in the scenario of FIG. 5(a), a test strip 502 that has received a “complete” fill with fluid herein is represented as line 503, fully covering each of the first and second working electrodes WE₁ and WE₂. FIG. 5(b) illustrates a scenario in which the first working electrode WE₁ is fully covered with fluid, but the second working electrode WE₂ is only partially covered with fluid, as represented by line 505. FIG. 5(c) illustrates an advanced low volume scenario in which the first working electrode WE₁, as represented by line 507. In the latter scenario and after a predetermined amount of time (e.g., 500 milliseconds), the processor is programmed to annunciate an error if the second working electrode remains substantially uncovered with sample fluid.

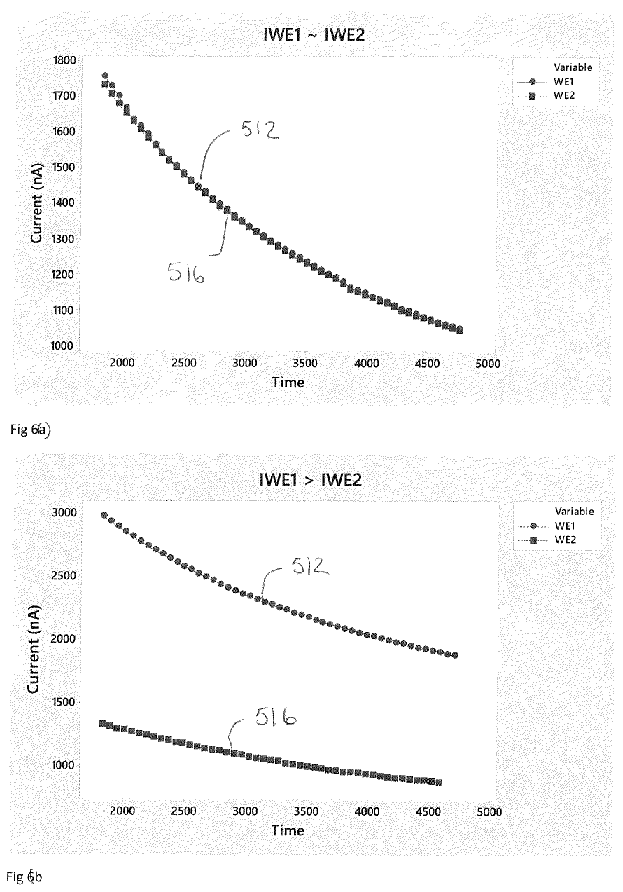

Representative current transients for each of the foregoing scenarios of FIGS. 5(a) and 5(b) are shown graphically in FIGS. 6(a) and 6(b), respectively, based on time following the initiation of a testing sequence. As shown in FIG. 6(a), there is a nearly identical 1:1 correspondence between the measured current transients of the first and second working electrodes I_{WE1} and I_{WE2}, respectively, herein depicted by reference numerals 512 and 516. On the other hand and in the case of only partial coverage of the second working electrode WE₂ as shown in FIG. 6(b), there is a considerable offset between the measured current transient for the first working electrode I_{WE1}, shown as 512, and the measured current transient for the second working electrode I_{WE2}, shown as 516.

An improved low volume logic is now described for an analyte (glucose) measurement system using the described test strip 100 or variants thereof having two or more working electrodes WE₁ and WE₂ as used with a test meter, mainly in regard to the scenario of FIG. 5(a). It should be noted that for the following methodology, the first and second working electrodes are planarly disposed as previously discussed and are of the same planar size relative to one another.

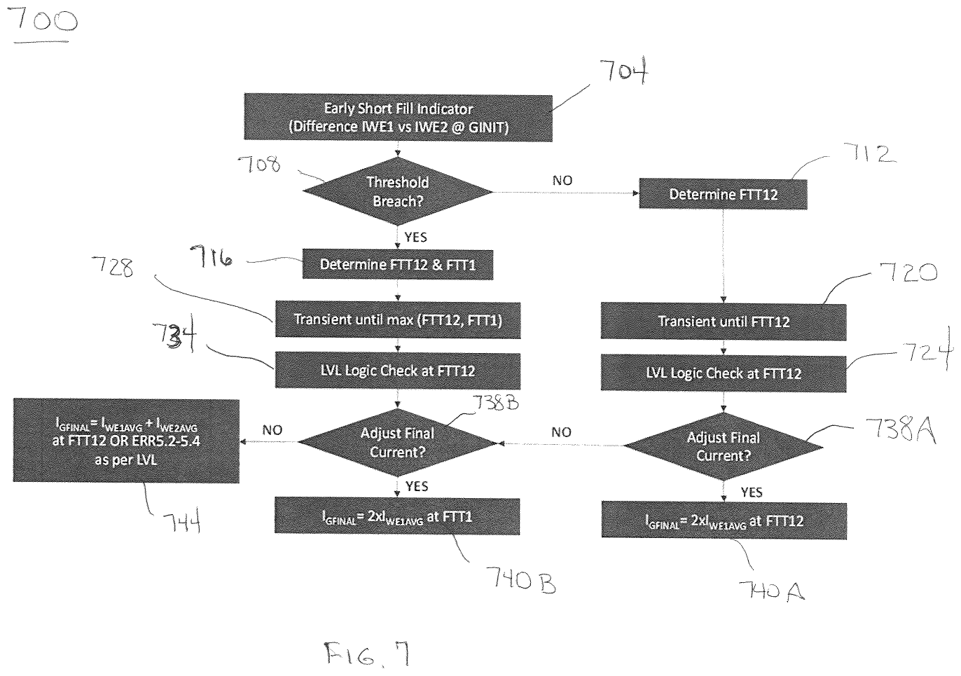

A flowchart 700 for an exemplary low volume logic (LVL) is provided at FIG. 7, as programmed into the processor of the test meter, such as test meter 200, FIG. 2. Preferably, the test meter programmed with logic for use with the test strip 100 previously described (or appropriate variants, see 502, FIG. 5(a). More specifically and at step 704 and following the deposit of a fluid sample onto the test strip, an early “short fill” of the test strip (or its variants) can be determined by measuring the difference between the currents at the first and second working electrodes (I_{WE1} and I_{WE2}) at a predetermined first time or time point following the initiation of the test sequence, herein referred to as G_{INIT}. According to this exemplary embodiment, this latter time point is representative of an initial glucose measurement time. For the test strip 100 and with prior reference to FIG. 4, G_{INIT} occurs at about 2.5 seconds from the initiation of the test sequence (that is, from the time the fluid sample is deposited onto the test strip).

At step 708, the difference in measured current between the first and second working electrodes [(I_{WE1}−I_{WE2})/I_{WE1}] at the predetermined first time point is compared to a predetermined threshold value or range of values, which is stored in the memory of the processor of the test meter. According to at least one version, this threshold can be between 0.1 and 0.4, and more preferably between 0.2 and 0.3, but it will be understood that this parameter can be suitably varied. If the difference in measured currents between the first and second working electrodes [(I_{WE1}−I_{WE2})/I_{WE1}×100] at the predetermined first time point G_{INIT} is determined to be less than the stored threshold, which would not constitute a threshold breach per step 708, the processor is then programmed at step 712 to determine a final test time, herein referred to as FTT₁₂, based on the sum of the measured currents of the first and second working electrodes I_{WE1} and I_{WE2} at the predetermined first time point.

On the other hand and if current difference exceeds the stored threshold; that is, a threshold breach following step 708 is determined, then the processor is then further programmed, at step 716, to determine two (2) final test times, namely, a first final test time (FTT₁), which is based only upon the measured current of the first working electrode I_{WE1} and a second final test time (FTT₁₂), which as previously noted is based on the sum of the measured currents of the first working electrode and second working electrodes (I_{WE1}+I_{WE2}) at the predetermined test time point (approximately 2.5 seconds). According to this exemplary embodiment, each of the final test times FTT₁ and FTT₁₂ can be derived from a look up table that is stored in the memory of the test meter that uses initial glucose estimate calculated using I_{WE1} and I_{WE2} currents at G_{INIT} and impedance, the latter depending on the hematocrit level in the blood sample, as determined by the physical characteristic sensing electrodes of the test strip. The foregoing is described in greater detail in U.S. Pat. No. 9,423,374 B2, which is herein incorporated by reference in its entirety.

Referring to the scenario in which there is no threshold breach following step 708, and specifically following the determination of the final test time FTT₁₂ per step 712, the current transients of the first and second working electrodes I_{WE1} and I_{WE2} are measured and stored at predetermined intervals (e.g., at 60 millisecond intervals, though this parameter can be suitably varied) from the predetermined first time point G_{INIT} until the final test time FTT₁₂, per step 720. According to this exemplary embodiment and at the same time, the processor is also preferably programmed to check the overall shape or integrity of the measured current transient of the first working electrode I_{WE1}, in order to prevent use of a corrupted current transient. Techniques for determining the shape and integrity of the current transient are described in greater detail in a later portion of this description.

Still referring to the flow chart of FIG. 7, and in the instance in which a threshold breach of the short fill is determined at step 708, then both final test time options FTT₁₂ and FTT₁ are determined at step 716. As noted, the final test times FTT₁ and FTT₁₂ can be derived according to this exemplary embodiment from a look up table stored in the memory of the test meter that uses initial glucose estimate calculated using I_{WE1} and I_{WE2} currents at G_{INIT} and impedance, the latter depending on the amount of hematocrit in the blood sample as determined by the physical characteristic sensing electrodes of the test strip using impedance, as described in U.S. Pat. No. 9,423,374 B2, which is herein incorporated by reference in its entirety. More specifically, FTT₁₂ uses both I_{WE1} and I_{WE2} currents. while FTT₁ uses only the I_{WE1} current, which is multiplied by two according to this logic, with the two working electrodes being planarly disposed and of the same relative size. At step 728, the current transient is measured at predetermined intervals until the greater of either FTT₁₂ or FTT₁ as determined by the processor wherein each of the current transients will be stored.

Once the longer (greater) of the two final test times FTT₁₂ or FTT₁ is determined per step 728, a low volume logic check is made at FTT₁₂ as per step 734. More specifically and according to steps 724 and 734 of the herein described method, a low volume logic (LVL) check at the final test time FTT₁₂. According to this exemplary embodiment, the processor of the test meter is programmed to first compare the measured current transients of the first and second working electrodes I_{WE1} and I_{WE2} with one another, and following this comparison is further programmed depending on the comparison to perform separate determinations of the current transients based on shape and other biasing, as herein discussed. More specifically, the difference in measured currents at FTT₁₂ between the first and second working electrodes WE₁ and WE₂ are compared to one another per the relation [(I_{WE1}−I_{WE2})/I_{WE1}].

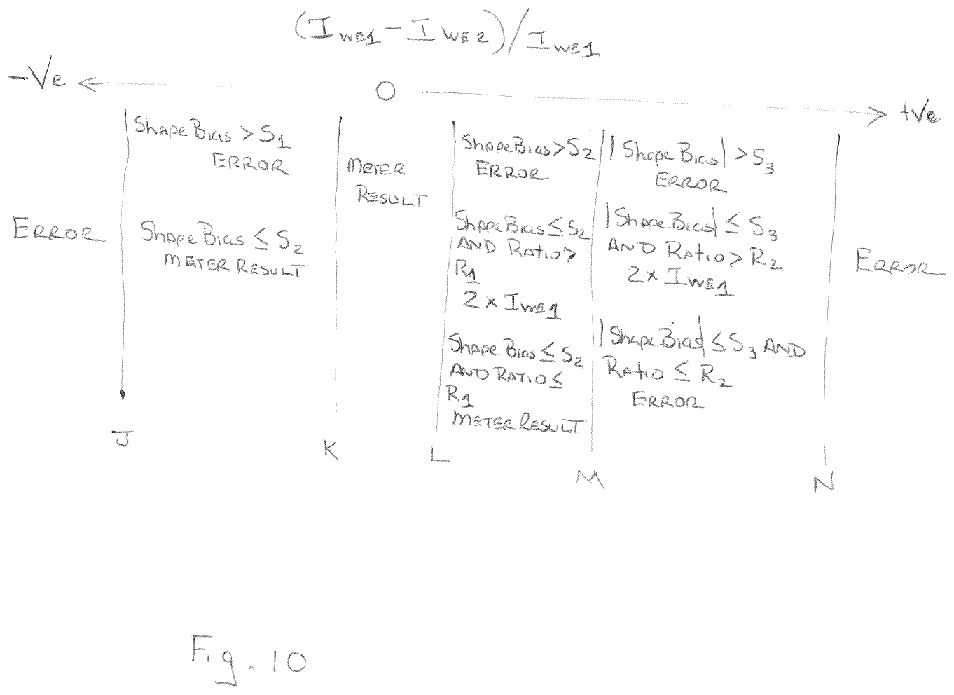

An exemplary logic matrix 1000 is provided at FIG. 10, including a spectrum that is dependent upon the compared current differences between the first and second working electrodes at the predetermined time, the latter being computed as percentages. Ranges of the measured percentages are further denoted as J, K, L, M and N at the bottom of the matrix 1000, including different required actions, each depending on the measured percentage. In the exemplary logic matrix, the following are used:
J, K from −30 to −15%
L, M from 15 to 30%
N from 70 to 95%
   It will be understood, however, that the above-listed percentages are provided for illustrative purposes and can be suitably varied. In this example, the above ranges have been multiplied by 100 in order to yield a percentage.

As noted, the processor is further programmed to perform various shape and bias checks, depending on the current differences noted above, and as provided in the logic matrix 1000. The nature of these shape and bias checks are now briefly described in general, and then as part of the overall low volume logic, steps 724, 734 by way of examples.

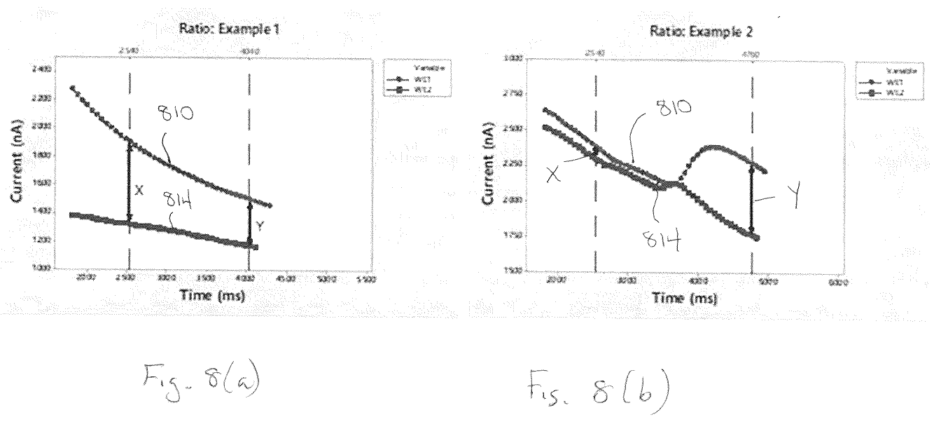

First and with reference to FIGS. 8(a) and 8(b), exemplary current transients 810 and 814 are shown as representative of I_{WE1} and I_{WE2}, respectively, with measured current being presented in nanoamperes over time, which is presented in milliseconds at predetermined intervals between the first time point (G_{INIT}) and the final test time (FTT₁₂), each of the latter times being shown by vertical dashed lines. According to one integrity check, differences between the current transients 810, 814 can be determined by the processor of the test meter, which can be configured as shown to determine a ratio X/Y of the measured current differences between the first and second working electrodes (I_{WE1}−I_{WE2}), at each of the first and second test times (i.e., G_{INIT} and FTT₁₂), respectively. The determined ratio between the current differences at the first predetermined time point G_{INIT} and the final test time FTT₁₂ can be applied, as needed, according to the exemplary logic matrix 1000 of FIG. 10 in which specific ranges of R1, R2 are set forth for purposes of the integrity check. According to this specific embodiment, R1 and R2 can vary between 50 to 100 0.

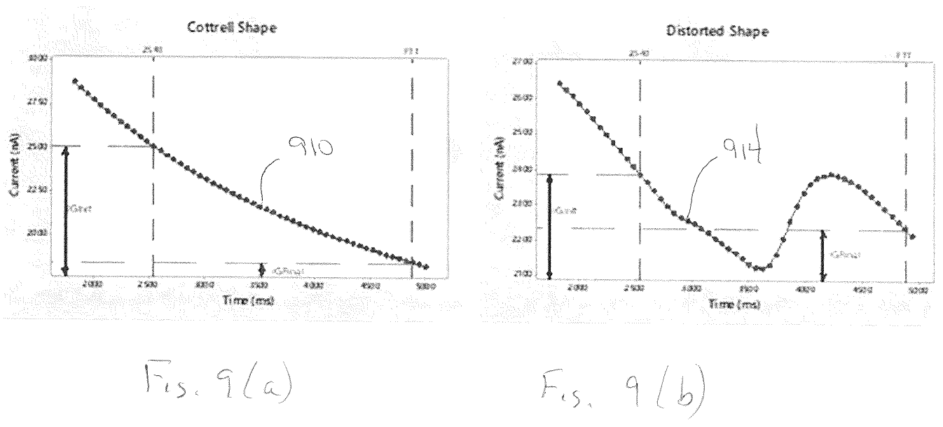

In addition and as part of the logic check performed at steps 724 and 734, the current transients I_{WE1} and I_{WE2} can also be measured in terms of a shape bias, such as shown in FIGS. 9(a) and 9(b). In this shape check, a transient shape can be evaluated or compared with that of a typical or standard Cottrell current distribution shape between the first and second predetermined test times. As in the preceding, each of the current transients, shown here as 910 and 914, depict current as measured in nanoamperes versus time, as measured in milliseconds. For purposes of illustration, a standard Cottrell transient 910 is depicted in FIG. 9(a) and a “distorted” current transient is shown in FIG. 9(b). For the exemplary transients shown, the following shape parameters can be defined:

$${SHAPE 1 = ( {I}_{WE 1} , {G}_{INIT} ) \times {G}_{INIT} / {G}_{FINAL} )}^{1 / 2}$$

$${SHAPE 2 = ( {I}_{we 2} , {G}_{INIT} ) \times {G}_{INIT} / {G}_{FINAL} )}^{1 / 2}$$

$$SHAPE 1 BIAS = [ ( SHAPE 1 - ( {I}_{we 1} , {G}_{FINAL} ) ) / ( {I}_{we 1} , {G}_{FINAL} ) ] \times 100$$

$$SHAPE 2 BIAS = [ ( SHAPE 2 - ( {I}_{WE 2} , {G}_{FINAL} ) ) / ( {I}_{WE 2} , {G}_{FINAL} ) ] \times 100$$

$$SHAPEBIAS ( S ) = MAX ( \vert SHAPE 1 BIAS \vert . \vert SHAPE 2 BIAS \vert )$$

in which G_{INIT} is the time point, G_{FINAL} is the final time point (e.g., FTT₁₂ or FTT₁); I_{WE1}, G_{INIT} is the first working electrode current at time G_{INIT}; I_{WE2} G_{INIT} is the second working electrode current at time G_{INIT}, I_{WE1}, G_{FINAL} is the first working electrode current at time G_{FINAL} and I_{WE2}, G_{FINAL} is the second working electrode current at time G_{FINAL}.

The resulting ShapeBias (S) can be applied as needed in accordance with the exemplary logic matrix 1000, FIG. 10, in which three (3) enumerated shape bias, namely S1, S2 and S3, vary between 10 to 60%.

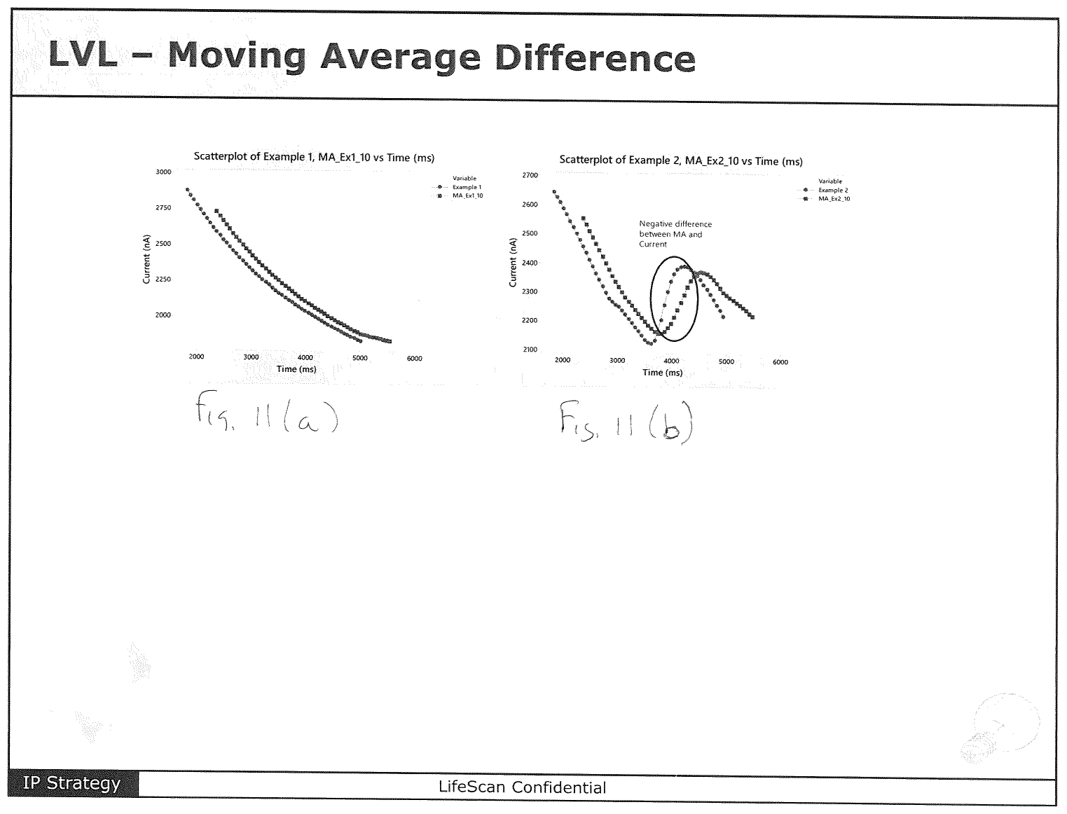

According to yet another exemplary variation and as shown in the scatterplots provided at FIGS. 11(a) and 11(b), another integrity check that can be used in place of or in combination with the foregoing checks is to identify a difference between the moving average and actual measured current of the transients, which can also be used to determine a shape distortion between the current transients of the first and second working electrodes of the test strip.

As noted, FIG. 10 presents an exemplary logic matrix 1000 embodying the aspects of the herein low volume logic performed at steps 724 and 734 to determine if the final current (IG_{final}) as measured at FTT₁₂ should be adjusted, per steps 738A or 738B, FIG. 7. The underlying logic of the matrix 1000 is initially based on the difference between the currents of the first and second working electrodes ((I_{WE1}−I_{WE2})/I_{WE1}×100 percent) at the final test time FTT₁₂ (or the higher of FTT₁ or FTT₁₂) and the previously described shape and integrity checks, including ratio and shape biasing. In the embodiment shown, the measured difference (measured as a percentage of the first working electrode current) in current triggers whether additional integrity or shape checks are required in making a low volume logic assessment or alternatively, whether an error should be annunciated without further recourse. For example and if the measured current difference at the final test time is less than “J” or greater than “N”, then an error is annunciated and the test sequence is terminated. Each of the foregoing represent extreme differences indicative of a serious under or overfill condition.

Sample scenarios are now described to illustrate the application of the low volume logic at steps 724 and 734, FIG. 7, based on the matrix 1000, FIG. 10, in order to determine whether the final current should be adjusted or an error should be annunciated by the analyte measurement system. In a first scenario, a threshold determination between the measured currents of the first and second current transients I_{WE1} and I_{WE2} at FTT₁₂ is determined to be zero. In this scenario, then the questions at each of the steps 738A and 738B in the flowchart 700, FIG. 7, are answered in the negative as “NO” and the processor is programmed to step 744, in which no adjustment is used for analyte measurement. The foregoing therefore relates “Meter Result” as indicated between points K and L in the matrix 1000 of FIG. 10. Accordingly, no shape or bias checks are required and the meter reading can be used with no adjustment of the final current value.

In a second scenario, the compared percentage between the first and second working electrode is determined to be −20 percent, which is a negative value as shown between J and K on the logic matrix 1000, FIG. 10. As such, the current of the second working electrode I_{WE2} is larger than that of the first working electrode I_{WE1}, which is not an indication of a potential low volume fill since fluid sample must encounter the first working electrode WE₁ before the second working electrode WE₂. In this scenario, no ratio comparison is required, but the processor is further programmed to determine shape checks of the current transient. According to the matrix 1000 and if the shape bias is greater than S1, then an error is annunciated. If on the other hand, the shape bias is equal to or less than S2, then the meter result is used.

In a third scenario, the measured percentage between the currents is about 20 percent, placing the percentage value between the L and M portions of the logic matrix 1000. This measurement requires additional integrity and shape checks being performed as programmed into the processor of the test meter. More specifically and if the shape bias is greater than S2, an error is annunciated. Alternatively and if the shape bias is less than or equal to S2 and the ratio (X/Y) is greater than R1, then the resulting current should be adjusted to 2×I_{WE1}. If the shape bias is less than or equal to S2 and the ratio is less than or equal to R1, then the meter result is used. Those practiced in the art may appreciate that certain of the relationships referred to throughout this disclosure and according to this methodology are based on two working electrodes of equal dimension or planar area, and so will be influenced by differences in the relative dimensions of the 2 working electrodes.

Finally and according to a fourth scenario, the current difference between the first and second working electrodes at the final test time is determined to be approximately 60 percent, which is between the M and N portions of the logic matrix 1000, FIG. 10. Accordingly, the following is determined. First and if the absolute value of the shape bias is greater than S3, then an error is annunciated and the test is terminated. Alternatively and if the absolute value of the shape bias is less than or equal to S3 and the ratio is greater than R2, then the current is adjusted to 2×I_{WE1}. Finally and if the absolute value of the shape bias is less than or equal to S3 and the ratio is less than or equal to R2, then an error is annunciated and the test is terminated.

Overall and if a determination is made that the final measured current IG_{final} does require adjustment, then the final current is adjusted as I_{GFINAL}=2×I_{WE1AVG} at FTT₁₂ per step 740A or as IG_{final}=2×I_{WE1Avg} at FTT1 per step 740B. If the final current does not require adjustment, per step 744, then I_{GFINAL}=I_{WE1AVG}+I_{WE2AVG} at FTT₁₂ or an error is annunciated based on ratio or shape bias differences, as previously discussed.

### PARTS LIST FOR FIGS. 1-11(b)

5 substrate
7 reference electrode track
9 second working electrode track
10 reference electrode
10a shielding/grounding electrode
11 reference contact pad
12 first working electrode
13 first contact pad
14 second working electrode
15 second contact pad
16 insulation layer
16′ insulation layer
17 strip detection bar
19a physical characteristic measurement electrode
19b third electrode track
20a physical characteristic measurement electrode
20b fourth electrode track
22a reagent layer
22b reagent layer
22 reagent layers
22′ reagent layer
24 adhesive portion (first adhesive pad)
26 adhesive portion (second adhesive pad)
29 spacer
32 distal hydrophilic portion
34 hydrophilic film layer
38 top layer
28 adhesive portion
50 (first) conductive layer
60 adhesive layer
70 hydrophilic layer
80 top layer
92 sample receiving chamber/test chamber
92a entrance, sample receiving chamber
92b opposite end, sample receiving chamber
95 physiological fluid sample
100 biosensor (test strip)
102 display
106 strip port connector
108 USB interface
112 microcontroller block
114 physical characteristic measurement block
116 display control block
118 memory block
200 test meter
204 display
206 first user interface input
210 second user interface input
214 third user interface input
218 data port
220 strip port connector
300 processor (microcontroller)
302 memory
304 ASIC (Application Specific Integrated Circuit)
306 analog interface
308 core
310 ROM
312 RAM
316 A/D converter
318 clock
320 display driver
502 test strip
503 fluid fill
505 fluid fill
507 fluid fill
512 first working electrode, current transient
516 second working electrode, current transient
700 flow chart, logic
702 current transient
704 step
708 step
712 step
716 step
720 step
724 step
728 step
734 step
738A step
738B step
740A step
740B step
744 step
810 current transient
814 current transient
910 current transient
914 current transient
1000 logic matrix
WE₁ first working electrode
WE₂ second working electrode
I_{WE1} current transient—first working electrode
I_{WE2} current transient—second working electrode
FTT₁ final test time
FTT₁₂ final test time
R ratio or ratio bias
S shape or shape bias

It will be understood that various modifications and variations can be made within the intended scope of the invention. For example, the preceding discussion relied upon a test strip having two working electrodes and in which the first and second working electrodes are defined by the same surface area and in which only two working electrodes are provided. It will be understood that the herein described method is also applicable to working electrodes having different surface areas in which suitable correction factors can be adjusted.

1. An analyte measurement system comprising:
a test strip comprising a first working electrode and a second working electrode in spaced relation, said electrodes being of the same relative planar size; and
a test meter including a processor configured to apply one or more test voltages to the test strip after a fluid sample has been deposited, wherein the processor is programmed to:
a) measure current levels at a predetermined time point following the initiation of a test sequence at each of the first and second working electrodes;
b) compare a difference in the measured current values at the first and second working electrodes at the predetermined time point to a predetermined threshold;
c) if the difference in the measured current values is within the predetermined threshold at the predetermined time point, then a final test time is determined based on the measured current of the first and second working electrodes; and
d) if the difference in the measured current values is not within the predetermined threshold, then two final test times are determined, a first final test time being based on the measured current values of both the first and second working electrodes and a second final test time being based on a suitably corrected measured current of only the first working electrode.

2. The measurement system of claim 1, wherein the processor is further programmed to:
e) measure and store current transients until the larger of the first or second final test times such that if the current difference at the predetermined time point is within the predetermined threshold, then the processor is programmed to only determine the second final test time.

3. The measurement system of claim 2, in which the processor is further configured to determine a shape bias between the current transients of the first and second working electrodes.

4. The measurement system of claim 3, in which the shape bias is based on a ratio difference in measured current taken at two predetermined test times between the first and second working electrode.

5. The measurement system of claim 3, in which the shape bias is based on a shape difference in relation to a Cottrell current transient.

6. The measurement system of claim 4, in which the processor is further programmed to determine the difference in measured currents between the first and second working electrode at the final test time.

7. The measurement system of claim 6, wherein the processor is programmed to compare the difference in measured currents between the first and second working electrode at the final test time and the difference in measured currents between the first and second working electrode at the first predetermined time point and to determine a ratio between the differences in current.

8. A method for compensating the measurement of an analyte of interest in a fluid sample on a test strip having two or more spaced working electrodes of substantially the same planar size based on low fluid volume on one of the spaced working electrodes, the method comprising:
a) depositing the fluid sample on the test strip;
b) applying one or more test voltages to the two or more spaced working electrodes;
c) measuring current at a first working electrode and a second working electrode of the test strip at a predetermined time after deposit of the fluid sample;
d) comparing a difference in the measured current at the first working electrode and the second working electrode to a stored threshold; and
e) if the difference in the measured current fails to meet the stored threshold, then calculating the analyte concentration in the deposited fluid sample at a final test time based on the current measured at the first working electrode and if the difference in the measured current meets the stored threshold, then calculating the analyte concentration based on a summing of the currents of the first and second working electrodes at a final test time.

9. The method according to claim 8, wherein the final test time is determined by a look up table stored in the test meter, the look up table being further based on hematocrit in the fluid sample, as determined by hematocrit sensing electrodes separately provided on the test strip.

10. The method according to claim 8, further comprising the step of determining differences in shapes between a current transient between the first and second test times and a predetermined transient shape.

11. The method according to claim 10, wherein the predetermined transient shape is a Cottrell transient shape.

12. The method according to claim 8, further comprising measuring a ratio of current differences between the first and second current transients at a first predetermined test time and a second predetermined test time.

13. A method for compensating an analyte measurement in the event of a low sample volume of a fluid sample being deposited onto a test strip having at least first and second working electrodes and a reference electrode in spaced relation, the first and second working electrodes being planarly disposed and of substantially the same size, the method comprising:
depositing the fluid sample onto the test strip;
applying at least one test voltage between the first and second working electrodes and the reference electrode;
using a processor, measuring current at each of the first and second working electrodes at a predetermined time point;
using the processor, comparing the difference in current measured at the first working electrode with the current measured at the second working electrode at the predetermined time point relative to a stored threshold; wherein if the difference in currents measured between the first working electrode and the second working electrode at the predetermined time point is within the stored threshold, then using the current measurements of the first and second working electrodes in determining an analyte of interest at a final test time; and if the difference in measured currents between the first working electrode and the second working electrode is not within the stored threshold at the predetermined time point, then using only the measured current of the first working electrode with a suitable correction applied in determining the analyte of interest at the final test time.

14. The method according to claim 13, further comprising, using the processor, measuring current transients of the first and second working electrodes between the predetermined time point and a final test time.

15. The method according to claim 14, wherein if the difference in the measured currents between the first working electrode and the second working electrode is not within the stored threshold, then determining two final test times in which one of the final test times is determined based on the current of the first and second working electrodes and the other final test time is based only on the current of the first working electrode.

16. The method according to claim 15, further comprising measuring current transients until the longer of the final test times.

17. The method according to claim 16, further comprising determining the difference in measured currents between the first and second working electrodes at the final test time.

18. The method according to claim 16, further comprising the step of determining differences in shapes between a current transient measured between the predetermined time point and the final test time and a predetermined current transient shape.

19. The method according to claim 18, wherein the predetermined transient shape is that of a Cottrell distribution.

20. The method according to claim 13, further comprising measuring a ratio of current differences between the first and second current transients at a first predetermined test time and a second predetermined test time.
